(19) United States
(12) Patent Application Publication
(10) Pub. No.: US 2004/0182134 A1
(43) Pub. Date: Sep. 23, 2004

(54) Gas chromatograph with fast BTU analysis

(76) Inventors: Stephen Staphanos, Long Beach, CA (US); Po-Liang Chien, Rancho Palos Verdes, CA (US)

Correspondence Address: WESTMAN, CHAMPLIN & KELLY, A PROFESSIONAL ASSOCIATION, SUITE 1600 - INTERNATIONAL CENTRE, 900 SECOND AVENUE SOUTH, MINNEAPOLIS, MN, 55402-3319, US

(21) Appl. No.: 10/390,345

(22) Filed: Mar. 17, 2003

(51) Int. Cl. G01N 30/02

(52) U.S. Cl. 73/23.35

(57) ABSTRACT

A gas chromatograph is provided for measuring energy content of a sample. The gas chromatograph includes a sample handling system enclosure and an analyzer enclosure. The sample handling system enclosure has a sample inlet port to receive a sample from a sample probe. The analyzer enclosure contains a first sample loop having a first volume, a second sample loop having a second volume, and a third sample loop having a third volume. Each of the first, second and third sample loops are operably coupled to the sample inlet port. First, second and third analysis columns are disposed within the analyzer enclosure and operably coupled to first, second and third sample loops, respectively. A thermal conductivity detector is disposed in the analyzer enclosure and includes a first portion operably coupled to the first column, and a second portion operably coupled to the second and third columns. The thermal conductivity detector provides signals indicative of thermal conductivity of each portion.

### BACKGROUND OF THE INVENTION

The present invention relates to gas chromatography. More particularly, the present invention relates to a gas chromatography apparatus that determines BTU content of a sample.

Gas chromatographs are frequently used to measure physical properties of natural gas. Gas chromatographs (GC's) can measure relative concentrations of a number of the primary-natural gas components and the measured component concentrations can thereafter be used to calculate important parameters such as energy content (hereinafter referred to as BTU). Since natural gas is a source of energy, the energy content of the natural gas itself is a very important quantity for both suppliers and purchasers of natural gas. For example, a purchaser of natural gas will generally pay more for the same quantity of natural gas if it has a higher energy content.

During the processing or transportation of natural gas, it is often useful to monitor the relative concentrations of the components of the natural gas. Should the relative concentrations of components of the natural gas change, appropriate parties can be notified. Further, since the dew point of natural gas is determined primarily by the concentration of the C₉+, it is important to monitor that concentration in order to detect dew point changes. Since remedial action regarding a detected condition can only be taken after the detection is complete, it is generally beneficial to provide BTU analysis as quickly as possible. One manner in which the gas chromatography of natural gas BTU analysis has been hastened, is by adding additional GC channels, where a sample is directed into a pair of GC modules. Each module is specifically tailored to detect components above or below a molecular threshold and provide an output thereof. In this manner, GC cycle time can be substantially reduced, but this reduction is accompanied by a significant increase in equipment costs. Specifically, each GC module generally includes all of its own valving, an independent CC oven, as well as an independent detector, such as a thermal conductivity detector. Thus, there is a current need to provide a GC system that provides fast BTU analysis while also reducing, or minimizing the costs of such system.

### SUMMARY OF THE INVENTION

A gas chromatograph is provided for measuring energy content of a sample. The gas chromatograph includes a sample handling system enclosure and an analyzer enclosure. The sample handling system enclosure has a sample inlet port to receive a sample from a sample probe. The analyzer enclosure contains a first sample loop having a first volume, a second sample loop having a second volume, and a third sample loop having a third volume. Each of the first, second and third sample loops are operably coupled to the sample inlet port. First, second and third analysis columns are disposed within the analyzer enclosure and operably coupled to first, second and third sample loops, respectively. A thermal conductivity detector is disposed in the analyzer enclosure and includes a first portion operably coupled to the first column, and a second portion operably coupled to the second and third columns. The thermal conductivity detector provides signals indicative of thermal conductivity of each portion.

### BRIEF DESCRIPTION OF THE DRAWINGS

FIG. 1 is a diagrammatic view of a process gas chromatography system in which embodiments of the present invention are particularly useful.

FIG. 2 is a diagrammatic view of a gas chromatography system in accordance with embodiments of the present invention.

FIG. 3 is a chromatogram produced from a GC system in accordance with an embodiment of the present invention.

### DETAILED DESCRIPTION OF THE PREFERRED EMBODIMENTS

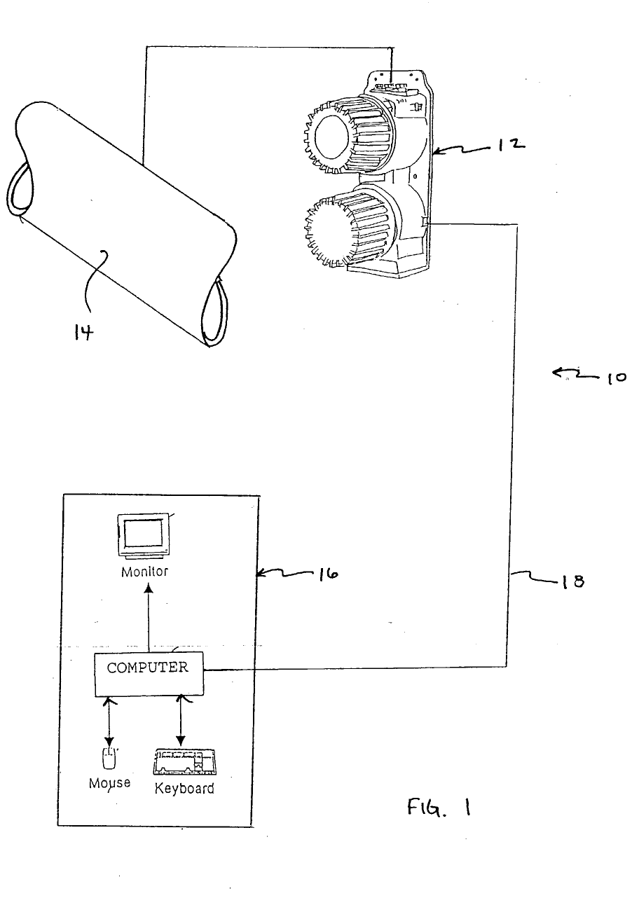

FIG. 1 is a diagrammatic view of a process gas chromatography system in which embodiments of the present invention are particularly useful. Process gas chromatography systems differ from laboratory gas chromatography systems in that the gas chromatography unit itself is generally contained within a field hardened enclosure. One example of such a process gas chromatograph is known as the Model GCX gas chromatograph available from the Rosemount Analytical Division of Emerson Process Management. FIG. 1 illustrates gas chromatography system 10 including process GC 12 operably coupled to a process fluid container, such as pipe 14, and to computer 16. A sample of process fluid, in this case natural gas, in pipe 14 is conveyed to GC 12. The sample stream is analyzed by GC 12 to provide information for calculating the BTU content of the natural gas flowing through pipe 14 as well as other meaningful information, as will be described in greater detail later in the specification. The output of GC 12 is provided to computer 16, which analyzes the GC data to actually generate the BTU analysis and other relevant information.

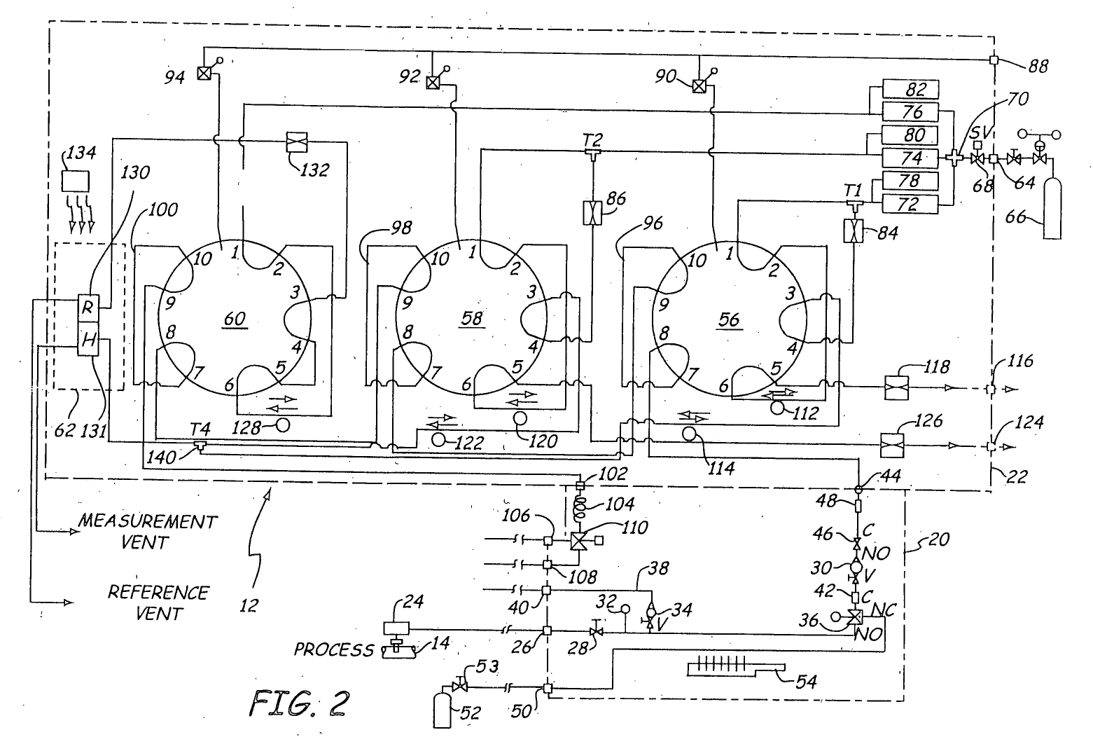

FIG. 2 is a diagrammatic view of process GC 12 in accordance with an embodiment of the present invention. GC 12 includes sample handling system enclosure 20 coupled to analyzer enclosure 22.

Sample handling system enclosure 20 is coupled to process 14 via sample probe 24. Sample probe 24 can be any suitable probe able to withdraw a portion of natural gas from pipe 14 and provide the natural gas to sample inlet 26 of sample handling system enclosure 20. Sample inlet 26 is coupled to valve 28, which is preferably a needle valve that allows a user to balance the flow to flow controller 30. Preferably, the flow is selected to be approximately 100 milliliters per minute. The output of valve 28 is also fluidically coupled to pressure indicator 32, bypass flow controller 34 and solenoid valve 36. Adjusting bypass flow controller 34 determines the amount of flow passing through lines 38 and out bypass vent port 40. Preferably, the bypass vent is constructed from one quarter inch outside diameter stainless steel. The common port of solenoid valve 36 is coupled to flow controller 30 through filter 42. Flow controller 30 is coupled to port 44 of analyzer enclosure 22 through solenoid valve 46 and filter 48.

Sample handling system enclosure 20 is also coupled, through port 50, to a source of calibration gas 52, through valve 54. Port 50 is fluidically coupled to solenoid valve 36 such that energization of valve 36 can selectively provide either sample gas or calibration gas to port 44 of analyzer enclosure 22. A thermal control system 54 is also disposed within sample handling system enclosure 20 and includes suitable components (not shown) such as a heat source, a temperature sensor, and a controller to maintain the interior of sample handling system enclosure 20 at an elevated temperature.

Analyzer enclosure 22 contains 10-port valves 56, 58, 60 and detector 62. Analyzer enclosure 22 is coupled, via carrier gas port inlet 64, to a source of carrier gas 66, which is preferably helium. Solenoid valve 68 is fluidically interposed between carrier gas inlet 64 and four-way tee 70. Tee 70 splits the carrier gas flowing from source 66 three ways with each stream passing into one of micromechanical flow valves 72, 74 and 76. One example of such a micromechanical flow valve is a silicon Fluistor microvalve available from Redwood Micro Systems of Menlow Park, Calif. The outputs of microvalves 72, 74 and 76 are fluidically coupled to pressure sensors 78, 80 and 82, respectively. Thus, the pressure sensor signals from sensors 78, 80 and 82 can be provided to a single controller (not shown) which can provide pressure signals to the appropriate microvalves in order to suitably maintain the pressures on the outlets of microvalves 72, 74 and 76. The outlets of microvalves 72, 74 and 76 are coupled to ports 1 of 10-port valves 56, 58 and 60, respectively. Further, the outputs of microvalves 72 and 74 are coupled to ports 4 of 10-port valves 56 and 58, respectively, through needle valves 84 and 86, respectively.

Instrument air, preferably having a pressure approximately 60 to 80 pounds per square inch gage (PSIG), is coupled to analyzer enclosure 22 at port 88. The instrument air is selectively conveyed to 10-port valves 56, 58 and 60 based upon energization signals provided to solenoid valves 90, 92 and 94, respectively. Conveying pressurized instrument air to a 10-port valve will cause the 10-port valve to switch from a first position to a second position. The two positions of the 10-port valves essentially let a given port be selectively coupled to either the neighboring port on its left or the neighboring port on its right. For example, FIG. 2 indicates ports 1 and 2 of 10-port valve 56 being coupled. A second position of 10-port valve 56 will couple ports 10 and 1 together. Thus, those skilled in the art will recognize FIG. 2 represents a state of valves 56, 58 and 60 in which all three valves are set in a first position.

In the state illustrated in FIG. 2 sample or calibration gas flowing through port 44 will pass into port 8 of 10-port valve 56; out port 7 of 10-port valve 56; through sample loop 96; into port 10 of 10-port valve 56; out port 9 of 10-port valve 56; into port 8 of 10-port valve 58; out port 7 of 10-port valve 58; through sample loop 98; into port 10 of 10-port valve 58; out port 9 of 10-valve 58; into port 8 of 10-port valve 60; out port 7 of 10-port valve 60; through sample loop 100; into port 10 of 10-port valve 60; out port 9 of 10-port valve 60; through port 102 on sample analyzer enclosure 22; through equilibrium coil 104; and finally out either atmospheric vent port 106 or sample return port 108 based upon a signal to solenoid valve 110.

The remaining port couplings of 10-port valves 56, 58 and 60 are set forth in the table below.

| Valve 56, Port 1 | Coupled to the output |
|---|---|
| | of microvalve 72. |
| Valve 56, Port 2 | Coupled to a first end |
| | of column 112. |
| Valve 56, Port 3 | Coupled to a first end |
| | of column 114. |
| Valve 56, Port 4 | Coupled to the output |
| | of microvalve 72 |
| | through needle valve |
| | 84. |
| Valve 56, Port 5 | Coupled to stripper |
| | vent output 116 |
| | through needle valve |
| | 118. |
| Valve 56, Port 6 | Coupled to a second |
| | end of column 112. |
| Valve 56, Port 7 | Coupled to a first end |
| | of sample loop 96. |
| Valve 56, Port 8 | Coupled to port 44. |
| Valve 56, Port 9 | Coupled to valve 58, |
| | port 8. |
| Valve 56, Port 10 | Coupled to the other |
| | end of sample loop 96. |
| Valve 58, Port 1 | Coupled to the output |
| | of microvalve 74. |
| Valve 58, Port 2 | Coupled to a first end |
| | of column 120. |
| Valve 58, Port 3 | Coupled to a first end |
| | of column 132, and |
| | ultimately to |
| | measurement portion |
| | 131 of detector 62. |
| Valve 58, Port 4 | Coupled to the output |
| | of microvalve 74 |
| | through needle valve |
| | 86. |
| Valve 58, Port 5 | Coupled to stripper |
| | vent port 124 through |
| | needle valve 126. |
| Valve 58, Port 6 | Coupled to the other |
| | end of column 120. |
| Valve 58, Port 7 | Coupled to a first end |
| | of sample loop 98. |
| Valve 58, Port 8 | Coupled to valve 56, |
| | port 9. |
| Valve 58, Port 9 | Coupled to Valve 60, |
| | port 8. |
| Valve 58, Port 10 | Coupled to the other |
| | end of sample loop 98. |
| Valve 60, Port 1 | Coupled to the output |
| | of microvalve 76. |
| Valve 60, Port 2 | Coupled to a first end |
| | of column 128. |
| Valve 60, Port 3 | Coupled to thermal |
| | conductivity detector |
| | reference portion 130 |
| | via needle valve 132. |
| Valve 60, Port 4 | Coupled to valve 60, |
| | port 5. |
| Valve 60, Port 5 | Coupled to valve 60, |
| | port 4. |
| Valve 60, Port 6 | Coupled to the other |
| | end of column 128. |
| Valve 60, Port 7 | Coupled to a first end |
| | of sample loop 100. |
| Valve 60, Port 8 | Coupled to valve 58, |
| | port 9. |
| Valve 60, Port 9 | Coupled to port 102. |
| Valve 60, Port 10 | Coupled to the other |
| | end of sample loop |
| | 100. |

Preferably, columns 112, 114, 120, 122 and 128 are constructed as follows. Column 112 is a “stripper” column preferably having a length of approximately one foot and an outside diameter of one eighth inch. Column 112 is preferably constructed from stainless steel and is packed with any suitable packing. Column 112 is conditioned by subjecting it to a temperature of approximately 220° C. for approximately four hours. Column 114 is an analysis column having a length of approximately two feet. Column 114 preferably has an outside diameter of one eighth inch and is also constructed from stainless steel. Preferably, column 114 constructed using the same packing and conditioning as column 112, described above. Column 120 is a commercially available stripper column formed using a stainless steel capillary. Column 120 preferably has dimensions of approximately 15 meters by 0.53 millimeter outside diameter by 5.0 micrometer inside diameter. Column 122 is an analysis column and is preferably constructed and configured identically to column 120 described above. Column 128 is an analysis/backflow column that is preferably constructed of the same materials and of the same dimensions as that set forth above with respect to columns 122 and 120.

The relative volumes of the samples loops are varied in order to provide an appropriate amount of sample to their respective columns. Sample loop 96 is preferably sized to contain a sample size of approximately 40 microliters. Sample loop 98 is preferably sized to contain a sample size of approximately 250 microliters, while sample loop 100 is preferably sized to contain a sample volume of 1,000 microliters.

Thermal conductivity detector 62 is disposed within enclosure 22, and includes reference portion 130 and measurement portion 131. Detector is thermally coupled to thermal control system 134 to maintain detector 62 at a temperature above that of the interior of enclosure 22. Preferably, detector 62 is maintained at a temperature at least about five degrees Centigrade above the temperature within enclosure 22.

Instead of using reference portion 130 as a true reference, embodiments of the present invention employ reference portion 130 as a second detector. Thus, with relatively minor alterations, a conventional GC can be configured to be a dual channel GC without using multiple GC ovens, and multiple thermal conductivity sensors. This provides the benefits of a dual-channel GC without the added costs of an additional GC oven, or multiple thermal conductivity detectors. Moreover, since embodiments of the present invention can be practiced by relatively straightforward modifications to current gas chromatographs, it is believed that industry adoption of products embodying features of the present invention will be facilitated.

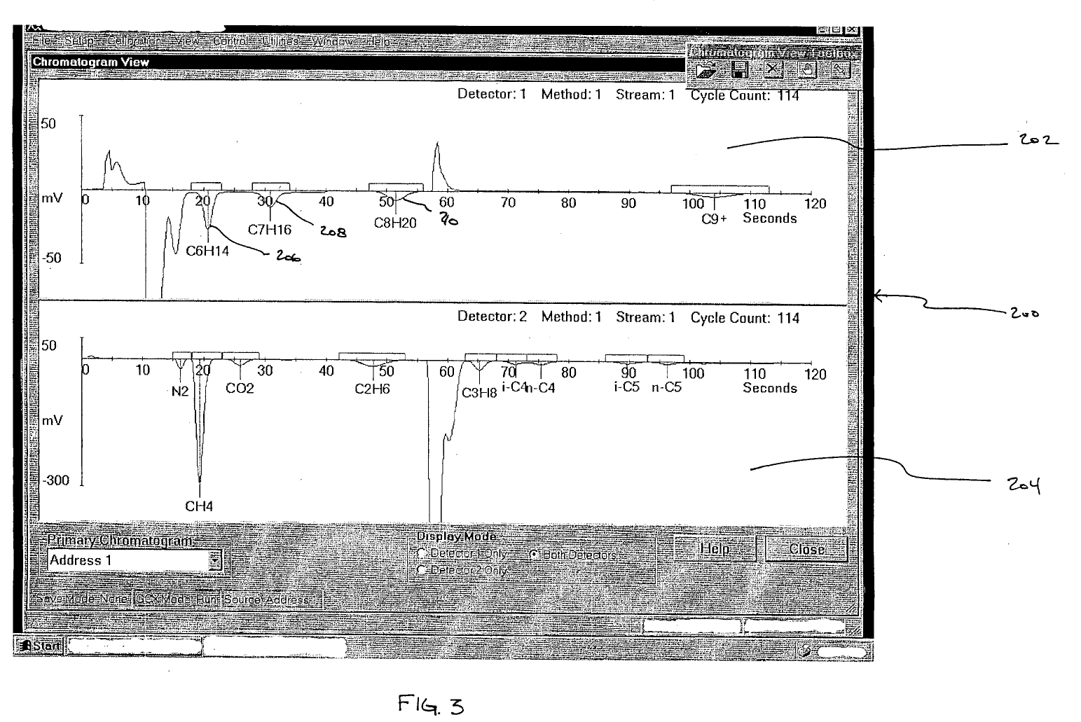

Operation of GC 12 occurs as follows. With reference to the time line illustrated in FIG. 3, valve 56 is energized to inject sample at 3 seconds (injection is a state of the 10 port valve in which the following port couplings occur: 10-1; 2-3; 4-5; 6-7; and 8-9), and to enter a stripping mode at around 40 seconds. This will detect, by virtue of column 114, relatively small molecular weight compounds such as N₂, CH₄, CO₂, and C₂H₆.

Valve 58 begins its injection at approximately 55 seconds, somewhat after the completion of the stripping operation of valve 56. At approximately 100 seconds, valve 58 enters a stripping mode and higher molecular weight compounds are eluted from column 122 for analysis. These higher molecular weight compounds include C₃H₈, IC₄, NC₄, IC₅, and NC₅. Those skilled in the art will recognize that operation of valves 56 and 58 occur essentially sequentially with one another providing the eluted compounds through T 140 at different times. “T” 140 provides the eluted compounds to measurement portion 131 on one side of the thermal-conductivity sensor 62.

Valve 60 essentially operates in parallel with valves 56 and 58. Specifically, valve 60 enters an injection mode at approximately 4 seconds and a back flush mode at approximately 58 seconds. This longer-term analysis allows elution and detection of the substantially higher molecular weight compounds such as C₆H₁₄, C₇H₁₆, C₈H₂₀, and C₉₊. The substantially higher molecular weight compounds are analyzed on reference portion 130 of thermal-conductivity detector 62. In this manner operation of GC 12 can be considered as occurring both serially (with respect to valves 56 and 58) and in parallel (with respect to the combination of valves 56 and 58; in parallel with vale 60). This allows extremely fast analysis of the various constituents of the sample gas while also providing useful resolution with respect to the ability to discern many different constituents over a relatively large range of molecular weights.

FIG. 3 is a chromatogram produced from a GC system in accordance with an embodiment of the present invention. A top portion 202 of chromatogram 200 shows the response of detector 1, which can be reference portion 130 (shown in FIG. 2). Upper portion 202 illustrates detection of the C₆+(heavies). Peak 206 indicates hexane C₆H₁₄, while peaks 208 and 210 indicate heptane C₇H₁₆ and octane C8H20, respectively. Peak 212 indicates the concentration of C₉+and FIG. 3 illustrates that this peak was observed within two minutes of the beginning of the analysis.

Bottom portion 204 of chromatogram 200 shows the response of detector 2, which can be measurement portion 131 (shown in FIG. 2). Peaks indicating concentrations of various lighter molecules are shown in bottom portion 204. The pentane molecules also generate peaks on bottom portion 204 before two minutes have elapsed. Thus, combining the information from top portion 202, and bottom portion 204 allow a seamless chromatogram to be constructed for the relevant components of natural gas within two minutes. Further, by providing data indicative of the C9+concentration within two minutes, quicker detection of dew point changes in the natural gas may be possible with embodiments of the present invention.

Although the present invention has been described with reference to preferred embodiments, workers skilled in the art will recognize that changes may be made in form and detail without departing from the spirit and scope of the invention.

What is claimed is:

1. A gas chromatograph for measuring energy content of a sample, the system comprising:
a sample handling enclosure having a sample inlet port to receive a sample from a sample probe;
an analyzer enclosure containing a first sample loop having a first volume, a second sample loop having a second volume, and a third sample loop having a third volume;
wherein each of the first, second and third sample loops are operably coupled to the sample inlet port;
first, second and third analysis columns disposed within the analyzer enclosure and operably coupled to first, second and third sample loops, respectively;
a thermal conductivity detector disposed in the analyzer enclosure and having a first portion operably coupled to the first column, and a second portion operably coupled to the second and third columns, wherein the thermal conductivity detector provides signals indicative of thermal conductivity of each portion.

2. The gas chromatograph of claim 1, wherein the first, second and third sample volumes differ from one another.

3. The gas chromatograph of claim 1, wherein the first sample volume is approximately 1000 microliters.

4. The gas chromatograph of claim 3, wherein the first column is a stainless steel capillary column.

5. The gas chromatograph of claim 1, wherein the second sample volume is approximately 250 microliters.

6. The gas chromatograph of claim 5, wherein the second column is a stainless steel capillary column.

7. The gas chromatograph of claim 1, wherein the third sample volume is approximately 40 microliters.

8. The gas chromatograph of claim 1, and further comprising a first thermal control system coupled to the sample handling system enclosure and the analyzer enclosure to maintain the interiors of the enclosures at a first elevated temperature.

9. The gas chromatograph of claim 8, and further comprising a second thermal control system coupled to the thermal conductivity detector to maintain the thermal conductivity detector at a second elevated temperature in excess of the first elevated temperature.

10. The gas chromatograph of claim 9, wherein the second elevated temperature exceeds the first elevated temperature by at least five degrees centigrade.

11. The gas chromatograph of claim 1, wherein the first sample loop is operably coupled to the first column through a first ten-port valve disposed within the analyzer enclosure.

12. The gas chromatograph of claim 11, wherein the second sample loop is operably coupled to the second column through a second ten-port valve disposed within the analyzer enclosure.

13. The gas chromatograph of claim 12, wherein the third sample loop is operably coupled to the third column through a third ten-port valve disposed within the analyzer enclosure.

14. The gas chromatograph of claim 1, wherein the gas chromatograph is a process gas chromatograph.

15. The gas chromatograph of claim 1, wherein the first portion of the thermal conductivity detector registers a signal indicative of C₉+ within two minutes of initiation of analysis.
